(19) United States
(12) Patent Application Publication
(10) Pub. No.: US 2008/0011309 A1
(43) Pub. Date: Jan. 17, 2008

(54) Immobilization device

(76) Inventor: Keith Wehrung, Myrtle Beach, SC (US)

Correspondence Address: B. Craig Killough;Barnwell Whaley Patterson & Helms, LLC, P.O. Drawer H, Charleston, SC, 29402-0197, US

(21) Appl. No.: 11/484,096

(22) Filed: Jul. 11, 2006

(51) Int. Cl. A61B 19/00

(52) U.S. Cl. 128/869

(57) ABSTRACT

A container having a constraining strap present therein for attachment to an immobilization board. The container is attached to an underside, or bottom, of the immobilization board or similar device for transporting a person who is reclining on the board. Opposite ends of the straps pull from the ends of the container and are pulled into position to surround the patient. The opposing ends of the strap are secured by a fastener. After use, the container is pulled away from the bottom of the board so that the strap and container are removed. A pressure sensitive adhesive on top of the container is used to affix a new container to the underside of the board.

### FIELD OF THE INVENTION

This invention relates to constraining and restraining devices generally, and is more specifically directed to a constraining device for use in transportation of the injured or sick.

### BACKGROUND OF THE INVENTION

Immobilization boards are used to transport injured, and sometimes, sick people. Immobilization boards are characterized by top and bottom flat, or planar, surfaces, although some immobilization boards have rails that run the length of the board and which support the board when the board is positioned on a surface.

In use, an injured person is positioned on the board with minimal disturbance to the position of the injured person. The attending medical person, such as an emergency medical technician, positions the injured person on the board, then straps the person to the board. The person is retained on the board so that movement of the person relative to the board is minimalized, reducing or eliminating additional trauma during transportation of the injured person.

Currently, the straps are retained in place on the board by threading the straps through voids formed on opposite sides of the board. The straps are routed through the orifices or voids on the sides of the board, which maintains the position of the strap relative to the board. The strap holds and maintains the position of the injured person relative to the board. However, the voids in which the straps are positioned may not align with the patient in a way that anatomically provides optimum immobilization.

As with most medical procedures, cleanliness is important, although there are no current standards specifically relating to such straps. Still, infection should not be introduced to the injured person through the straps. Accordingly, the straps are disposed of after each use. Prior to use, the straps are positioned over the board and in position for use. By positioning the straps over the board prior to use, the straps are exposed to the surrounding environment, and are not protected from the surrounding environment, which may cause the straps to become soiled. The soil may carry with it harmful bacteria or other pathogens that could infect the patient. The straps may be positioned over the board for long intervals prior to use, furthering the likelihood of the introduction of harmful materials or pathogens to the straps prior to use.

There is a need for a device that will prevent the straps from being exposed to the environment, while at the same time properly position the straps for use. The device should provide for easy disposal of the straps after use, and easy replacement of new straps.

### SUMMARY OF THE PRESENT INVENTION

The present invention is a container having an immobilization strap that is present therein. The container is attached to an underside, or bottom, of an immobilization board or similar device for transporting a person who is reclining on the board. Opposite ends of the container may be easily accessed by the medical technician when the device is ready for use. Opposite ends of the straps pull from the ends of the container and are pulled into position to surround the patient. The opposing ends of the strap are secured by a fastener. After use, the container is pulled away from the bottom of the board so that the strap and container are removed. A pressure-sensitive adhesive on top of the container is used to affix a new container to the underside of the board.

### SUMMARY OF THE DRAWINGS

FIG. 1 shows an underside of an immobilization board, demonstrating three containers that are spaced apart for attachment to the underside of the board

FIG. 2 is an elevation of an end of an immobilization board showing a person positioned on the immobilization board, with the immobilization board and the container sectioned to demonstrate the position of the strap within the container prior to use.

FIG. 3 is a perspective view of a person strapped to an immobilization board using the present invention.

FIG. 4 shows the immobilization board with the container, and straps extending from the container, being removed from the immobilization board after use.

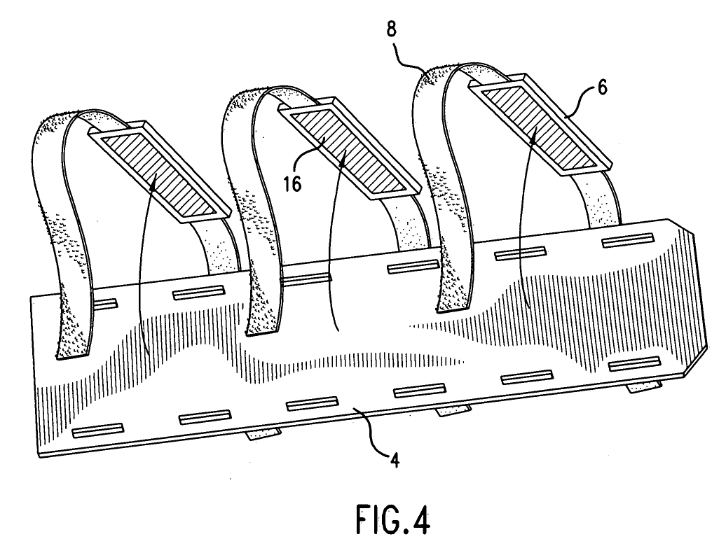

### DETAILED DESCRIPTION OF THE PREFERRED EMBODIMENTS

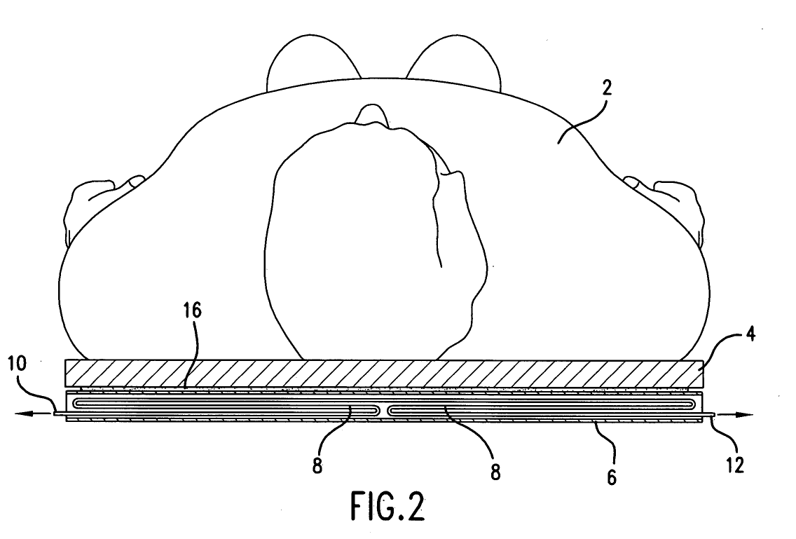

Referring now to the drawing figures, FIG. 2 shows a person 2 lying on an immobilization board 4. A conduit or container 6 for a strap 8 is affixed to the bottom, or underside of the immobilization board, and opposite the top surface upon which the person is positioned.

As shown in FIG. 2, a single strap is present within the container. The strap is a single, continuous strap in a preferred embodiment. The strap is folded for simple and orderly egress of the strap from the container. The strap has a first end 10 and a second end 12, the first end of the strap is adjacent to a first opening in a first end or side of the container. The second end of the strap is opposite the first end of the strap, and the second end of the strap is adjacent to a second opening on in a second end or side of the container. The end of the strap is positioned so that it is quickly and easily identified and grasped, and due to the folding of the strap, when the end is pulled, the strap will quickly and easily exit the container.

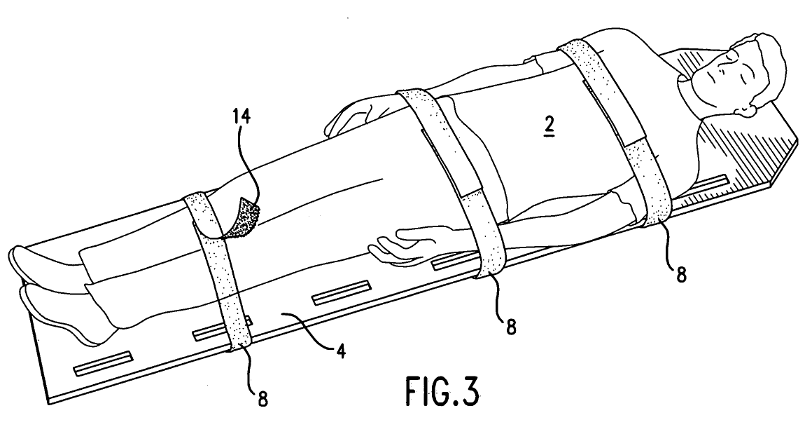

In use, the first end of the strap and the second end of the strap may be pulled simultaneously from the container and over the top of the board to surround the patient. The strap is completely, or substantially completely, retained within the container prior to use, so that the strap is not exposed to the environment, which could soil and/or contaminate the strap. In one embodiment, a small portion of the first end of the strap, and a small portion of the second end of the strap extend just outside the container for easy identification and grasping by the medical technician. After the first end of the strap and the second end of the strap are pulled, the portion of the strap which is outside of the container is sufficient to surround the patient, as shown in FIG. 3, while a portion of the strap, which is typically the center of the strap, is still retained within the container. When the device is in use, the container serves to assist in preventing the strap from moving longitudinally up and down by the board, by holding the strap in its position relative to the remaining straps and the board.

The straps may be formed of the same material as straps in current use. The strap has a fastener that allows the ends of the straps to be positioned over the patient and secured tightly to constrain and restrain the patient against the board. The fastener may be fasteners of types currently known and used to fasten belts and straps, such as loops, D-rings, and buckles. However, in a preferred embodiment, hook and loop material 14 is used, with one side of the material positioned on one strap and the associated material positioned on the opposing end of the strap. The hook and loop material provides a fast and secure means for securing the ends of the strap, and using the strap to secure the patient tightly against the immobilization board.

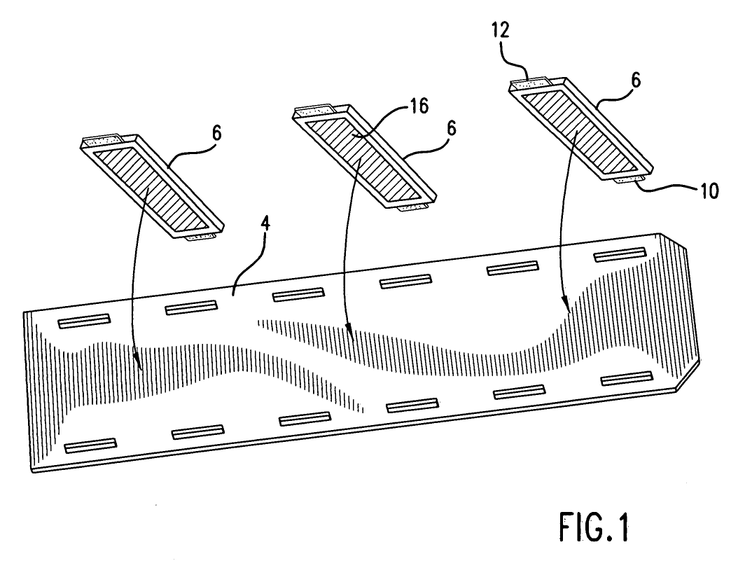

In use, an immobilization board may have a relatively flat bottom surface to which one or more containers are affixed. In most applications, it will be preferred to use three containers and straps, as shown in the drawing figures, although more or fewer containers and straps may be used as required. A top of the container has pressure-sensitive adhesive 16 thereon. Prior to use and storage, a protective sheet is applied over the pressure-sensitive material. This arrangement is similar to the arrangement used with bumper stickers. The protective sheet is peeled away from the top of the container, exposing the pressure-sensitive material. The containers are then positioned along the board in a spaced apart manner as shown in FIG. 1. The containers may be positioned as shown in FIG. 3, so that one strap surrounds a patient across the upper chest; another strap surrounds the patient near the lower torso; and, a third strap surrounds the patient at about the knees. The device allows the straps to be positioned anatomically on the patient so that optimum immobilization is achieved. In the event that the bottom of the container is not flat, an appropriate shaped filler member may be positioned between the board and the container. The filler may have pressure sensitive adhesive on one or both sides.

At least a portion of the bottom of the immobilization board is a relatively flat, hard surface, which is an appropriate receiver for the pressure-sensitive adhesive. The pressure-sensitive adhesive should be applied so as to securely hold the container in place as the board is moved about. However, the pressure-sensitive adhesive material should not be of such quantity or quality that it would be difficult to remove the container after the straps have been deployed. The technician should be able to remove the container from the board, without the use of tools or solvents. The technician replaces the used container and straps with one or more unused containers, each having a strap therein. Alternatively, the technician places a strap in position through the container, which acts as a conduit, just prior to deployment.

After the patient is positioned on top of the immobilization board, the strap is in position for deployment. Each end of the strap is pulled, preferably in relatively simultaneous fashion, by the emergency medical technician, so that the strap is fully extended for use. After the strap is fully extended for use, the strap is wrapped around the patient as shown in FIG. 3. The hook and loop material is present on the end of the straps, and is positioned so that the hook and loop material firmly and securely holds the patient against the immobilization board.

After use, the containers, with straps extended, are peeled away from the board, and discarded. This cycle is then repeated with new containers affixed, as shown in FIG. 1, having straps present within the containers.

The container has a top surface with pressure-sensitive material thereon. On either end of the container, there is an opening which allows access to the strap. The container, in one embodiment, may have a flap that is capable of being moved aside to permit access to the end of the strap. The flap in this embodiment is present on each end of the container, so that access is permitted to each end of the strap. In another embodiment, a small portion of each end of the strap extends from an opening in opposite ends of the container. The small portion is just large enough to allow the technician to grasp each end quickly without searching for the ends. The opening should be just large enough to allow the strap to pass through the opening without interfering with egress of the strap from the container. A small opening helps protect the strap from being soiled as it is being retained in the container. A membrane that is easily torn away may be used to protect the end of the strap in another embodiment, so that the container is sealed, and a sterile environment is maintained. In another embodiment, the straps are present in a container, such as a sealed bag, which may be plastic, and the straps are removed from the bag and positioned for use.

The container only need be slightly wider than the strap, so that the strap is folded within the container as shown in FIG. 2, and the container becomes a conduit for the strap about the bottom of the immobilization board when the strap is in use The length of the container is preferred to be substantially as wide as the immobilization board, so that the opposite ends of the container that are adjacent to the side of the immobilization board, and from which the opposing ends of the strap are emitted, may be quickly and easily identified by the emergency medical technician, without the technician having to search underneath the board for the ends of the container, and the ends of the strap. In one embodiment, the container may extend just beyond the sides of the immobilization board, but it is not preferred that the ends of the container not extend materially beyond the sides of the board, so that the ends of the container are not damaged as the immobilization board is transported prior to use.

The container may be formed of materials from which containers are commonly constructed, including, cardboard, or similar materials comprising cellulose, or from plastic, or textile materials. The container may be constructed so that the weight of the patient and the immobilization board rest on the container. In most embodiments, it is preferred that the container have sufficient rigidity to support the weight of the patient and the board. However, in some embodiments materials such as textile materials, which do not typically have such rigidity, may be used.

What is claimed is:

1. A constraining device, comprising:
a) a container, wherein said container has a pressure sensitive adhesive on a surface thereof, wherein said pressure sensitive adhesive is covered by a protective sheet of material prior to attachment of said container to a board;
b) a strap that is located within said container, said strap having a first end and a second end, wherein a length of said strap is sufficient to surround a girth of an adult human; and
c) a fastener for said strap, wherein said fastener connects said first end of said strap to said second end of said strap.

2. A constraining device as described in claim 1, wherein said strap is continuous from said first end to said second end.

3. A constraining device as described in claim 1, wherein said container comprises a first end, a second end and a top, and wherein said first end of said container is opposite said second end of said container, and said first end of said container is capable of movement relative to said top to access an interior of said container and said second end of said container is capable of movement relative to said top to access said interior of said container, and wherein said first end of said strap is positioned near said first end of said container and said second end of said strap is positioned near said second end of said container, and wherein said pressure sensitive adhesive is applied to said top of said container.

4. A constraining device as described in claim 1, wherein said fastener is hook and loop material.

5. A constraining device as described in claim 1, further comprising said board, wherein said board is not less than 1.75 meters in length, and said container is affixed to said board.

6. A constraining device as described in claim 5, wherein said top surface of said container is positioned against a bottom of said board by contact of said pressure sensitive adhesive against said board after said protective sheet is removed from said pressure sensitive material.

7. A constraining device as described in claim 6, wherein said container may be removed from said board by manual force and without the assistance of tools.

8. A constraining device as described in claim 6, further comprising a second container, wherein said second container has a pressure sensitive adhesive on a surface thereof, wherein said pressure sensitive adhesive is covered by a removable protective sheet of material prior to attachment of said second container to a board;
b) a strap for said second container that is located within said second container, said strap for said second container having a first end and a second end, wherein a length of said strap for said second container is sufficient to surround a girth of an adult human; and
c) a fastener for said strap for said second container, wherein said fastener for said strap for said second container connects said first end of said strap for said second container to said second end of said strap for said second container.

9. A constraining device as described in claim 8, wherein said pressure sensitive adhesive is on a top surface of said second container and said top surface of said second container is positioned against a bottom of said board by contact of said pressure sensitive adhesive of said second container against said board after said protective sheet is removed from said pressure sensitive material of said second container, and wherein said second container is spaced apart from said container.

10. A constraining device as described in claim 1, wherein, prior to use of said strap is surrounding said board, said strap is located entirely within said container, and during use of said strap is surrounding said board, a portion of said strap is located within said container, and said strap is not attached to said container prior to use of said strap or during use of said strap.
